(19) United States
(12) Patent Application Publication
(10) Pub. No.: US 2019/0269628 A1
(43) Pub. Date: Sep. 5, 2019

(54) ROOM TEMPERATURE STORAGE STABLE TRIENTINE COMPOSITION

(71) Applicant: CRONUS RESEARCH LABS PRIVATE LIMITED, Hyderabad (IN)

(72) Inventor: Satya Srinivas Chetlapalli, Bangalore (IN)

(73) Assignee: CRONUS RESEARCH LABS PRIVATE LIMITED, Hyderabad (IN)

(21) Appl. No.: 16/292,008

(22) Filed: Mar. 4, 2019

(30) Foreign Application Priority Data
Mar. 5, 2018 (IN) 201841008118

(51) Int. Cl. A61K 31/132; A61K 9/00; A61J 1/00

(52) U.S. Cl. CPC A61K 31/132; B65D 81/266; A61J 1/00; A61K 9/0056

(57) ABSTRACT

The invention relates to a room temperature storage stable trientine composition comprising of pharmaceutically acceptable solid oral dosage form of trientine; and oxidation resistant container system. The invention also relates to a method of storing trientine solid oral composition at room temperature without degradation.

### TECHNICAL FIELD OF THE INVENTION

The invention in general relates to pharmaceutical dosage forms containing trientine hydrochloride. Particularly, the invention relates to pharmaceutical composition of trientine hydrochloride that is stable at room temperature.

### BACKGROUND ART

Trientine, also known as Triethylenetetramine, is a chelating agent and is used to bind the excess copper by forming a stable complex with the copper, which is then removed from the body by urinary excretion.

Trientine hydrochloride capsules 250 mg is approved by USFDA and marketed as Syprine in USA for treatment of patients with Wilson's disease who are intolerant to Penicillamine.

The present formulations of trientine are unstable at room temperature and therefore have to be stored under refrigeration (2-8° C.) to prevent degradation.

### SUMMARY OF THE INVENTION

Herein disclosed is a room temperature storage stable trientine composition. The room temperature storage stable trientine composition comprises of pharmaceutically acceptable solid oral dosage form of trientine; and oxidation resistant container system; wherein the said room temperature storage stable trientine composition is stable without refrigeration up to a temperature of 25° C.

The room temperature storage stable trientine composition as disclosed herein comprises of oxidation resistant container system comprising of pharmaceutically acceptable container closure system; and one or more, oxygen absorbers or molecular sieves. The container closure system is selected from the group consisting of glass containers, PET containers, Plastic polymer containers or HDPE containers. Alternatively the container closure system comprises of a coating of molecular sieve on the inner layer of the container.

Further, the invention discloses a method of storing trientine solid oral composition at room temperature without degradation comprising of packaging the trientine solid oral composition in pharmaceutically acceptable container-closure system along with one or more oxygen absorbers or one or more molecular sieves in an inert atmosphere that excludes oxygen and moisture. Alternatively, according to the method of storing trientine solid oral composition as disclosed herein, the container closure system comprises of a coating of molecular sieve on the inner layer of the container.

### DETAILED DESCRIPTION OF THE INVENTION

Disclosed herein is a room temperature storage stable trientine composition which does not require refrigeration at 2-8° C. The room temperature storage stable trientine composition comprises of pharmaceutically acceptable solid oral dosage form of trientine; and oxidation resistant container system; wherein the said room temperature storage stable trientine composition is stable without refrigeration up to a temperature of 25° C.

According to one embodiment the oxidation resistant container system comprises of pharmaceutically acceptable container closure system; and one or more oxygen absorbers. The oxygen absorber comprises of iron powder, natural zeolite, salt and water and is packaged in a laminated film comprising of multiple layers. One or more oxygen absorbers can be included in the container-closure system along with the pharmaceutically acceptable solid oral dosage form of trientine. For example, 3 number of oxygen absorber sachets can be included in the container closure system along with the pharmaceutically acceptable solid oral dosage form of trientine.

According to another embodiment the oxidation resistant container system comprises of pharmaceutically acceptable container closure system; and one or more molecular sieves. The molecular sieves are composed of crystalline metal aluminosilicates wherein the aluminosilicates have three dimensional interconnecting network of silica and alumina tetrahedra. The size of molecular sieve can be between 4 to 8 mesh, depending on the size and material of the container-closure system. One or more molecular sieves can be included in the container-closure system along with the pharmaceutically acceptable solid oral dosage form of trientine. For example, 3 numbers of molecular sieves can be included in the container closure system along with the pharmaceutically acceptable solid oral dosage form of trientine.

The container closure system is selected from the group consisting of glass containers, PET containers, Plastic polymer containers or HDPE containers.

According to another embodiment the container closure system comprises of a coating of molecular sieve on the inner layer of the container.

The invention disclosed herein also provides a method of storing trientine solid oral composition at room temperature without degradation comprising of packaging the trientine solid oral composition in pharmaceutically acceptable container-closure system along with one or more oxygen absorbers or one or more molecular sieves in an inert atmosphere that excludes oxygen and moisture.

According to one embodiment the inert atmosphere that excludes oxygen and moisture is created by purging nitrogen.

According to one embodiment the container closure system comprises of a coating of molecular sieve on the inner layer of the container.

We claim:

1. A room temperature storage stable trientine composition comprising of
a. pharmaceutically acceptable solid oral dosage form of trientine; and
b. oxidation resistant container system;
wherein the said room temperature storage stable trientine composition is stable without refrigeration up to a temperature of 25° C.

2. The room temperature storage stable trientine composition of claim 1, wherein the oxidation resistant container system comprises of
a. pharmaceutically acceptable container closure system; and
b. one or more oxygen absorbers.

3. The room temperature storage stable trientine composition of claim 1, wherein the oxidation resistant container system comprises of
a. pharmaceutically acceptable container closure system; and
b. one or more molecular sieves.

4. The room temperature storage stable trientine composition of claim 1, wherein the container closure system is selected from the group consisting of glass containers, PET containers, Plastic polymer containers or HDPE containers.

5. The room temperature storage stable trientine composition of claim 1, wherein the container closure system comprises of a coating of molecular sieve on the inner layer of the container.

6. A method of storing trientine solid oral composition at room temperature without degradation comprising of packaging the trientine solid oral composition in pharmaceutically acceptable container-closure system along with one or more oxygen absorbers or one or more molecular sieves in an inert atmosphere that excludes oxygen and moisture.

7. A method of storing trientine solid oral composition according to claim 6, wherein the container closure system comprises of a coating of molecular sieve on the inner layer of the container.
